SEQUENCE LISTING (3 sequences)

SEQ ID NO: 1 - DNA, 1032 bp, synthetic construct
  source 1..1032: mol_type = other DNA
atggctgatacaatgctagcggcagttgtacgcgaatttggtaagccgctgagcattgagcgcttgccgattccggatatcaaaccccatcagattctggtgaaggttgatacgtgcggtgtgtgtcataccgacctgcatgcagcaagaggcgactggccgagcaaaccgaacccgccatttatcccgggccacgaaggcgttggtcatattgtggcggtgggctcgcaagttggcgactttgttaaaaccggtgacgtggtcggcgtgccgtggctgtacagcgcgtgtggacactgtgagcactgcttgggtggttgggaaaccttgtgcgagaaacaggatgacacaggatacaccgtcaacgggtgcttcgcagaatacgtggtagcggatccgaattatgttgcgcacctgccttctactatcgatccgttacaggcttccccggttctctgcgctggcttgaccgtttacaaaggtctgaaaatgaccgaggcgcgtccgggccaatgggttgcggtttctggtgtgggtggcctaggccagatggcagtgcagtatgcggtcgctatgggtatgaacgtagtcgcggtggacatcgacgacgaaaagctggcgaccgcaaagaaactgggtgcctccctgacggtgaacgcgaaggataccgatccggctcgtttcatccagcaacagatcggcggtgcgcacggtgccctggtcaccgcggtcggtcgcaccgcgttcagccaagccatgggttatgcccgccgtggcggtacgatcgttctgaatgggctgccgccaggcgacttcccggtgtcgatcttcgacatggttatgaatggtacgaccattcgtggtagcatcgtgggcacccgtttggacatgattgaggcgatggatttctttgctcgtggcaaggtgaagagcgttgttactccgggtaagttagaaaatattaacaccatttttgatgacctgcaaaacggtcgtcttgagggccgtaccgttctggatttccgcagctaa

SEQ ID NO: 2 - DNA, 2316 bp, synthetic construct
  source 1..2316: mol_type = other DNA
atggcaaaaattgaacaaatagctaagaaatccgatgcgactcgcttgtctcgtagaaactttctgatgaccgcggcgggtgccggactgatgtttggcttcgcccgtaaagcaggcgcggcgaccaccttgccgtccgccatgccgcctgaggcggcgttcgagcccaacatttggtgtgcaatcgcacctgacggatccattaacgtgaacattgtgcgtgctgaaatgggccaacacgttggtaccgctctggcgcgaatcattgccgacgagatggatgcggactgggataaaattaagattacccaagttgacaccgcgccgaagtgggcaggtaaatatgttaccggtggcagctggtccgtctgggatacttgggacaccttccgtcaagcaggcgcagctgcgaggtctgtgatgattgaagaaggtgcgaagttgctcggtactacgccagatcgttgtaccgcccatgaaagcgttgttagcgcaggttcgaaatccatctcgttcggtgatatcgtggcgcgcgcgaagccgacccgcacatttaccccggaggagatggcgaagctgccgcttaagccgactggaaaccgccgtctgattagcaaacaggttccggctctcgatatcccggacaagacgaccggtaaggcgatttatggcatcgatgttaaattggacggcatggtctacggtcgtccgaagatgccgccaactcgctatgcggctaaggttattagcgtcgacgacagtgcagctaagaagattccgggctacctgcgttatgtggtcctggacgacccgtctggtattgtgccgggttgggttgtggcgctcgcgaaaacctacccggcggcgatccgtgcggcggatgccctgaaagtgcagtggaatccgggcccgaccatcaacgtcagcgaagcagatatcatcgagcatggtcggaagctggccgctgacccgaagaatggtacccgcgtttttaacgataagggtgtcgatgaggcattaaccatccacccgggtcaggtttttgagcgctcctatacctgcgcaagcgtggcgcattatcagttggagccggtcaatgccgtggctcgccacatcgacggcatgtgggaaattcacaccggcaaccagtggcagagcctgatcctgccacagctggctaagagcctgcaagttccggaagagcaggtggttatgcgtacctacatgctgggcggtggcttcggccgtcgtttaaacggcgattactgcattccggcggccctggcttcaaaggcgattggcggcgccccagttaaactaatactgacccgttctgatgacatggaactggacagcatccgttccccgtccatccaaacgatcaaagtggcgctggacaacgatcgtaagaaaatcgtgggtatggactacgtggcggtggcgggctggcctacgcaggtgatggcaccggcattcctggcgaccggcgaagatggcaaaaagtacgatccattcgctatcgctggcgcggatcattggtatgagaccggtccgacccgtgtgcgcgccatcagcaatgacctggcgaacgcaacgttccgcccgggttggctgagaagcgtatctgcaggttggaccccgtgggcattggagtgctttctggacgagttggcccacagcaccaaacaagatccgctggctttccgtcttagcatgttcaccgctcaaggtcgcaacgcgggacaagcaccgaacagcgtcggtggcgcgaaacgtcaggcggcggtgctgcagcgtttggccgacaaaatcggttacgcaaataaacaactgccggcggacaccggtattggtatcgccacgtccttcggccaagaaagaggtatgcccacttggaccgctgcggcggcacaaattcacgtggaccgcaaaaccggtgttgttacctgccagaaactgtggctggttctggatgcgggcacgattgtagatccgggtggcgctctggcgcagacggagggtgcggctttatggggtttcagcatggcattgtttgaaggtactgagatcgtcaacggcacgatcaaagatcgtaatctgaatacctacaccccgttgcgtattccggacgttccggacattgacatcgagtttattcagaataccgaaaagccgaccggcctcggtgaaccgggtgtaacggttgttgctccggctattggtaacgcgatctttaatgcggttggaattcgcttgcgccacatgccgatgcgtccggctgacgtgcgtcgtgaactgcaacagcataccagctaa

SEQ ID NO: 3 - DNA, 441 bp, synthetic construct
  source 1..441: mol_type = other DNA
atgctagatcagcaaacaattaatataataaaggcgacggtgccggttctgaaagagcacggcgtgaccattaccactaccttttacaaaaacttgtttgcaaaacatccggaagttcgtccgttgttcgacatgggtcgccaagagagcctggaacagccaaaggctctggctatgaccgttctcgcggcggcgcaaaatattgaaaacctgccggcaattctgccggcagttaagaagatcgcggtgaagcactgccaggcgggtgtggccgccgcgcattatccgatcgtgggtcaggagctgctgggcgcaatcaaagaggtcttgggcgacgctgctaccgatgatatcttagatgcttggggtaaagcgtatggcgttattgcagacgtgttcatccaggttgaggccgacctgtacgcgcaagcggtcgaataa

(19) United States
(12) Patent Application Publication
(10) Pub. No.: US 2026/0185127 A1
(43) Pub. Date: Jul. 2, 2026

(54) METHOD FOR PRODUCING GLYCOLIC ACID BY IMMOBILIZATION

(71) Applicant: NAN YA PLASTICS CORPORATION, TAIPEI (TW)

(72) Inventors: CHING-YAO YUAN, TAIPEI (TW); CHUNG-YU CHEN, TAIPEI (TW); YI-CHENG LI, TAIPEI (TW); Tzu-Huan Wong, Hualien County (TW); Liang-Jung Chien, TAIPEI (TW)

(21) Appl. No.: 19/081,348

(22) Filed: Mar. 17, 2025

(30) Foreign Application Priority Data
Dec. 30, 2024 (TW) 113151386

(51) Int. Cl. C12P 7/18; C07K 14/195; C12N 1/20; C12N 9/02; C12N 9/04; C12R 1/02; C12R 1/19

(52) U.S. Cl. CPC C12P 7/18; C07K 14/195; C12N 1/20; C12N 9/0006; C12N 9/0008; C12R 2001/02; C12R 2001/19; C12Y 101/01001; C12Y 102/01003

(57) ABSTRACT

A method for producing glycolic acid by immobilization includes: providing a modified strain, in which the modified strain includes gene sequences of SEQ ID NO: 1, SEQ ID NO: 2, and SEQ ID NO: 3; manufacturing a polyvinyl alcohol bacterial pellet, in which the polyvinyl alcohol bacterial pellet contains the modified strain; and supplying a carbon source to the polyvinyl alcohol bacterial pellet, so that the carbon source is converted into the glycolic acid by the modified strain. The polyvinyl alcohol bacterial pellet is mixed with an auxiliary solution, and air is introduced when the carbon source is supplied to the polyvinyl alcohol bacterial pellet.

### CROSS-REFERENCE TO RELATED PATENT APPLICATION

This application claims the benefit of priority to Taiwan Patent Application No. 113151386, filed on Dec. 30, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### STATEMENT REGARDING SEQUENCE LISTING

This application contains a sequence listing in XML format, which has been submitted electronically and is hereby incorporated by reference in its entirety. The sequence listing, created on Feb. 4, 2025, is named 113P001951US-SEQUENCELISTING.xml and is 7,077 bytes in size.

### FIELD OF THE DISCLOSURE

The present disclosure relates to a method for producing glycolic acid, and more particularly to a method for producing glycolic acid by immobilization.

### BACKGROUND OF THE DISCLOSURE

Glycolic acid (GA) is the smallest α-hydroxy carboxylic acid, and is also referred to as hydroxyacetic acid. The glycolic acid contains a carboxyl group and a hydroxyl group, thereby having dual properties of carboxylic acid and alcohol. Accordingly, the glycolic acid has characteristics of being easily degraded and absorbed, being highly water-soluble, and being penetrable. In addition, the glycolic acid and its polymers have good biodegradability and biocompatibility, and can be degraded and metabolized into water and carbon dioxide within an organism, so as to be discharged outside of the organism. As such, the glycolic acid has a wide range of application. For example, the glycolic acid and its polymers can be used to release polypeptides and protein drugs, or can act as pharmaceutical intermediates for ester preparation of menthol and quinine and synthesis of other pharmaceuticals. Glycolic acid oligomers or derivatives can be used in food additives, and reduce reproduction of harmful microorganisms by acidification.

In the conventional technology, the glycolic acid is mainly manufactured by chemical synthesis (e.g., formaldehyde cyanidation, chloroacetic acid hydrolysis, and formaldehyde-hydrogen hydroxylation). However, problems such as violent reaction conditions, highly toxic raw materials, high costs, difficulty in subsequent separation, and environment pollution are present in the chemical synthesis. Hence, enzyme catalysis of microorganisms and total biosynthesis are gradually developed to replace the chemical synthesis, and have become the main method for producing the glycolic acid.

It should be noted that the enzyme catalysis of microorganisms needs to use acetonitrile or glycolonitrile (hydroxyacetonitrile), which is toxic and costly as a raw material, and thus fails to satisfy the requirements of industrial production. The total biosynthesis is promising in the market of glycolic acid production, but mass application is still not possible due to yield limitation. Therefore, how to overcome the above-mentioned problems through process improvements has become one of the important issues to be solved in the relevant industry.

### SUMMARY OF THE DISCLOSURE

In response to the above-referenced technical inadequacies, the present disclosure provides a method for producing glycolic acid by immobilization.

In order to solve the above-mentioned problems, one of the technical aspects adopted by the present disclosure is to provide a method for producing glycolic acid by immobilization. The method includes: providing a modified strain, in which the modified strain includes gene sequences of SEQ ID NO: 1, SEQ ID NO: 2, and SEQ ID NO: 3; manufacturing a polyvinyl alcohol bacterial pellet, in which the polyvinyl alcohol bacterial pellet contains the modified strain; and supplying a carbon source to the polyvinyl alcohol bacterial pellet, so that the carbon source is converted into the glycolic acid by the modified strain. The polyvinyl alcohol bacterial pellet is mixed with an auxiliary solution, and air is introduced when the carbon source is supplied to the polyvinyl alcohol bacterial pellet.

In one of the possible or preferred embodiments, manufacturing the polyvinyl alcohol bacterial pellet includes: preparing a 1% to 20% polyvinyl alcohol solution, and adding the modified strain into the polyvinyl alcohol solution for uniform mixing, so as to form a polyvinyl alcohol bacterial solution; preparing a saturated boric acid solution and a phosphoric acid solution; dripping the polyvinyl alcohol bacterial solution into the saturated boric acid solution, so as to obtain a semi-finished product of the polyvinyl alcohol bacterial pellet; and taking out the semi-finished product of the polyvinyl alcohol bacterial pellet for draining, and pouring in the phosphoric acid solution and letting stand for a predetermined time, so as to obtain the polyvinyl alcohol bacterial pellet.

In one of the possible or preferred embodiments, manufacturing the polyvinyl alcohol bacterial pellet further includes preparing 0.5% to 1% sodium alginate.

In one of the possible or preferred embodiments, the predetermined time ranges between thirty minutes and two hours.

In one of the possible or preferred embodiments, a concentration of the saturated boric acid solution ranges between 1% and 5%, and a concentration of the phosphoric acid solution ranges between 0.1 M and 1 M.

In one of the possible or preferred embodiments, the carbon source is ethylene glycol, and an aeration volume of the air ranges between 0.3 vvm and 1 vvm.

In one of the possible or preferred embodiments, the modified strain is modified Escherichia coli, modified Gluconobacter sp., modified Rhodococcus, or modified oxidative bacilli.

In one of the possible or preferred embodiments, the auxiliary solution is formed by a yeast extract, peptone, sorbitol, (NH₄)₂SO₄, and MgSO₄·7H₂O.

In one of the possible or preferred embodiments, a GC % of each of the SEQ ID NO: 1, the SEQ ID NO: 2, and the SEQ ID NO: 3 ranges between 50% and 60%.

In one of the possible or preferred embodiments, the modified strain has an ability to produce an alcohol dehydrogenase, an aldehyde dehydrogenase, and vitreoscilla hemoglobin.

Therefore, in the method for producing the glycolic acid by immobilization provided by the present disclosure, by virtue of “providing a modified strain, in which the modified strain includes gene sequences of SEQ ID NO: 1, SEQ ID NO: 2, and SEQ ID NO: 3” and “manufacturing a polyvinyl alcohol bacterial pellet, in which the polyvinyl alcohol bacterial pellet contains the modified strain,” the technology of strain immobilization can be used to improve an efficiency of forming the glycolic acid by oxidation of the ethylene glycol.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a flowchart of a process for converting a carbon source into glycolic acid according to the present disclosure;

FIG. 2 is a schematic view of a plasmid according to the present disclosure;

FIG. 3 is a schematic view showing a gene sequence comparison between SEQ ID NO: 1 of the present disclosure and an existing alcohol dehydrogenase (EGADH);

FIG. 4 (FIG. 4A and FIG. 4B) is a schematic view showing a gene sequence comparison between SEQ ID NO: 2 of the present disclosure and an existing aldehyde dehydrogenase (EGAlDH);

FIG. 5 is a schematic view showing a gene sequence comparison between SEQ ID NO: 3 of the present disclosure and an existing vitreoscilla hemoglobin (VHb);

FIG. 6 is a curve and bar graph showing a conversion amount of the glycolic acid according to a first embodiment of the present disclosure;

FIG. 7 is a curve and bar graph showing the conversion amount of the glycolic acid according to a second embodiment of the present disclosure; and

FIG. 8 is a flowchart of a method for producing the glycolic acid by immobilization according to the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

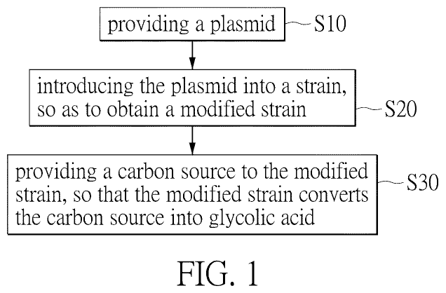

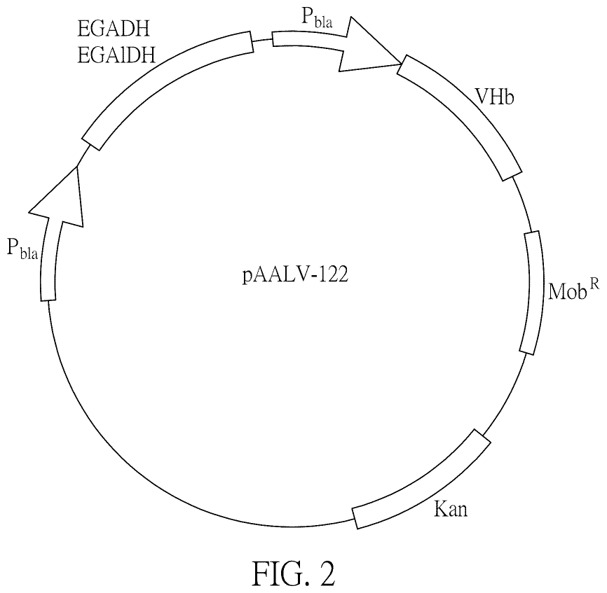

Referring to FIG. 1 and FIG. 2, the present disclosure provides a process for converting a carbon source into glycolic acid. The process includes: providing a plasmid (step S10), in which the plasmid includes gene sequences of SEQ ID NO: 1, SEQ ID NO: 2, and SEQ ID NO: 3; introducing the plasmid into a strain, so as to obtain a modified strain (step S20); and providing the carbon source to the modified strain, so that the modified strain converts the carbon source into the glycolic acid (step S30). Specifically, before step S10, the process can further include a step of synthesizing a DNA sequence. A sequence having genetic code identification that is suitable for a target strain can be artificially synthesized and provided for identification of the target strain, and a corresponding substance can be manufactured. In the present disclosure, a sequence having genetic code identification that is suitable for gram-negative bacteria is artificially synthesized. For example, the strain of the present disclosure can be Escherichia coli, Gluconobacter sp., Rhodococcus, or oxidative bacilli. However, the aforementioned examples describe only one of the embodiments of the present disclosure, and the present disclosure is not intended to be limited thereto.

In one embodiment of the present disclosure, the SEQ ID NO: 1 is a gene encoding an alcohol dehydrogenase (EGADH), or is a gene having a sequence identify of more than 80% with the SEQ ID NO: 1 and an activity of the alcohol dehydrogenase (EGADH). The SEQ ID NO: 2 is a gene encoding an aldehyde dehydrogenase (EGAIDH), or is a gene having a sequence identify of more than 80% with the SEQ ID NO: 2 and an activity of the aldehyde dehydrogenase (EGAIDH). The SEQ ID NO: 3 is a gene encoding vitreoscilla hemoglobin (VHb), or is a gene having a sequence identify of more than 80% with the SEQ ID NO: 3 and an activity of the vitreoscilla hemoglobin (VHb).

In the present disclosure, a sequence of the SEQ ID NO: 1 is:

ATGGCTGATACAATGCTAGCGGCAGTTGTACGCGAATTTGGTAAG
CCGCTGAGCATTGAGCGCTTGCCGATTCCGGATATCAAACCCCAT
CAGATTCTGGTGAAGGTTGATACGTGCGGTGTGTGTCATACCGAC
CTGCATGCAGCAAGAGGCGACTGGCCGAGCAAACCGAACCCGCCA
TTTATCCCGGGCCACGAAGGCGTTGGTCATATTGTGGCGGTGGGC
TCGCAAGTTGGCGACTTTGTTAAAACCGGTGACGTGGTCGGCGTG
CCGTGGCTGTACAGCGCGTGTGGACACTGTGAGCACTGCTTGGGT
GGTTGGGAAACCTTGTGCGAGAAACAGGATGACACAGGATACACC
GTCAACGGGTGCTTCGCAGAATACGTGGTAGCGGATCCGAATTAT
GTTGCGCACCTGCCTTCTACTATCGATCCGTTACAGGCTTCCCCG
GTTCTCTGCGCTGGCTTGACCGTTTACAAAGGTCTGAAAATGACC
GAGGCGCGTCCGGGCCAATGGGTTGCGGTTTCTGGTGTGGGTGGC
CTAGGCCAGATGGCAGTGCAGTATGCGGTCGCTATGGGTATGAAC
GTAGTCGCGGTGGACATCGACGACGAAAAGCTGGCGACCGCAAAG
AAACTGGGTGCCTCCCTGACGGTGAACGCGAAGGATACCGATCCG
GCTCGTTTCATCCAGCAACAGATCGGCGGTGCGCACGGTGCCCTG
GTCACCGCGGTCGGTCGCACCGCGTTCAGCCAAGCCATGGGTTAT
GCCCGCCGTGGCGGTACGATCGTTCTGAATGGGCTGCCGCCAGGC
GACTTCCCGGTGTCGATCTTCGACATGGTTATGAATGGTACGACC
ATTCGTGGTAGCATCGTGGGCACCCGTTTGGACATGATTGAGGCG
ATGGATTTCTTTGCTCGTGGCAAGGTGAAGAGCGTTGTTACTCCG
GGTAAGTTAGAAAATATTAACACCATTTTTGATGACCTGCAAAAC
GGTCGTCTTGAGGGCCGTACCGTTCTGGATTTCCGCAGCTAA.

A sequence of the SEQ ID NO: 2 is:

ATGGCAAAAATTGAACAAATAGCTAAGAAATCCGATGCGACTCGC
TTGTCTCGTAGAAACTTTCTGATGACCGCGGCGGGTGCCGGACTG
ATGTTTGGCTTCGCCCGTAAAGCAGGCGCGGCGACCACCTTGCCG
TCCGCCATGCCGCCTGAGGCGGCGTTCGAGCCCAACATTTGGTGT
GCAATCGCACCTGACGGATCCATTAACGTGAACATTGTGCGTGCT
GAAATGGGCCAACACGTTGGTACCGCTCTGGCGCGAATCATTGCC
GACGAGATGGATGCGGACTGGGATAAAATTAAGATTACCCAAGTT
GACACCGCGCCGAAGTGGGCAGGTAAATATGTTACCGGTGGCAGC
TGGTCCGTCTGGGATACTTGGGACACCTTCCGTCAAGCAGGCGCA
GCTGCGAGGTCTGTGATGATTGAAGAAGGTGCGAAGTTGCTCGGT
ACTACGCCAGATCGTTGTACCGCCCATGAAAGCGTTGTTAGCGCA
GGTTCGAAATCCATCTCGTTCGGTGATATCGTGGCGCGCGCGAAG
CCGACCCGCACATTTACCCCGGAGGAGATGGCGAAGCTGCCGCTT
AAGCCGACTGGAAACCGCCGTCTGATTAGCAAACAGGTTCCGGCT
CTCGATATCCCGGACAAGACGACCGGTAAGGCGATTTATGGCATC
GATGTTAAATTGGACGGCATGGTCTACGGTCGTCCGAAGATGCCG
CCAACTCGCTATGCGGCTAAGGTTATTAGCGTCGACGACAGTGCA
GCTAAGAAGATTCCGGGCTACCTGCGTTATGTGGTCCTGGACGAC
CCGTCTGGTATTGTGCCGGGTTGGGTTGTGGCGCTCGCGAAAACC
TACCCGGCGGCGATCCGTGCGGCGGATGCCCTGAAAGTGCAGTGG
AATCCGGGCCCGACCATCAACGTCAGCGAAGCAGATATCATCGAG
CATGGTCGGAAGCTGGCCGCTGACCCGAAGAATGGTACCCGCGTT
TTTAACGATAAGGGTGTCGATGAGGCATTAACCATCCACCCGGGT
CAGGTTTTTGAGCGCTCCTATACCTGCGCAAGCGTGGCGCATTAT
CAGTTGGAGCCGGTCAATGCCGTGGCTCGCCACATCGACGGCATG
TGGGAAATTCACACCGGCAACCAGTGGCAGAGCCTGATCCTGCCA
CAGCTGGCTAAGAGCCTGCAAGTTCCGGAAGAGCAGGTGGTTATG
CGTACCTACATGCTGGGCGGTGGCTTCGGCCGTCGTTTAAACGGC
GATTACTGCATTCCGGCGGCCCTGGCTTCAAAGGCGATTGGCGGC
GCCCCAGTTAAACTAATACTGACCCGTTCTGATGACATGGAACTG
GACAGCATCCGTTCCCCGTCCATCCAAACGATCAAAGTGGCGCTG
GACAACGATCGTAAGAAAATCGTGGGTATGGACTACGTGGCGGTG
GCGGGCTGGCCTACGCAGGTGATGGCACCGGCATTCCTGGCGACC
GGCGAAGATGGCAAAAAGTACGATCCATTCGCTATCGCTGGCGCG
GATCATTGGTATGAGACCGGTCCGACCCGTGTGCGCGCCATCAGC
AATGACCTGGCGAACGCAACGTTCCGCCCGGGTTGGCTGAGAAGC
GTATCTGCAGGTTGGACCCCGTGGGCATTGGAGTGCTTTCTGGAC
GAGTTGGCCCACAGCACCAAACAAGATCCGCTGGCTTTCCGTCTT
AGCATGTTCACCGCTCAAGGTCGCAACGCGGGACAAGCACCGAAC
AGCGTCGGTGGCGCGAAACGTCAGGCGGCGGTGCTGCAGCGTTTG
GCCGACAAAATCGGTTACGCAAATAAACAACTGCCGGCGGACACC
GGTATTGGTATCGCCACGTCCTTCGGCCAAGAAAGAGGTATGCCC
ACTTGGACCGCTGCGGCGGCACAAATTCACGTGGACCGCAAAACC
GGTGTTGTTACCTGCCAGAAACTGTGGCTGGTTCTGGATGCGGGC
ACGATTGTAGATCCGGGTGGCGCTCTGGCGCAGACGGAGGGTGCG
GCTTTATGGGGTTTCAGCATGGCATTGTTTGAAGGTACTGAGATC
GTCAACGGCACGATCAAAGATCGTAATCTGAATACCTACACCCCG
TTGCGTATTCCGGACGTTCCGGACATTGACATCGAGTTTATTCAG
AATACCGAAAAGCCGACCGGCCTCGGTGAACCGGGTGTAACGGTT
GTTGCTCCGGCTATTGGTAACGCGATCTTTAATGCGGTTGGAATT
CGCTTGCGCCACATGCCGATGCGTCCGGCTGACGTGCGTCGTGAA
CTGCAACAGCATACCAGCTAA.

A sequence of the SEQ ID NO: 3 is:

ATGCTAGATCAGCAAACAATTAATATAATAAAGGCGACGGTGCCG
GTTCTGAAAGAGCACGGCGTGACCATTACCACTACCTTTTACAAA
AACTTGTTTGCAAAACATCCGGAAGTTCGTCCGTTGTTCGACATG
GGTCGCCAAGAGAGCCTGGAACAGCCAAAGGCTCTGGCTATGACC
GTTCTCGCGGCGGCGCAAAATATTGAAAACCTGCCGGCAATTCTG
CCGGCAGTTAAGAAGATCGCGGTGAAGCACTGCCAGGCGGGTGTG
GCCGCCGCGCATTATCCGATCGTGGGTCAGGAGCTGCTGGGCGCA
ATCAAAGAGGTCTTGGGCGACGCTGCTACCGATGATATCTTAGAT
GCTTGGGGTAAAGCGTATGGCGTTATTGCAGACGTGTTCATCCAG
GTTGAGGCCGACCTGTACGCGCAAGCGGTCGAATAA.

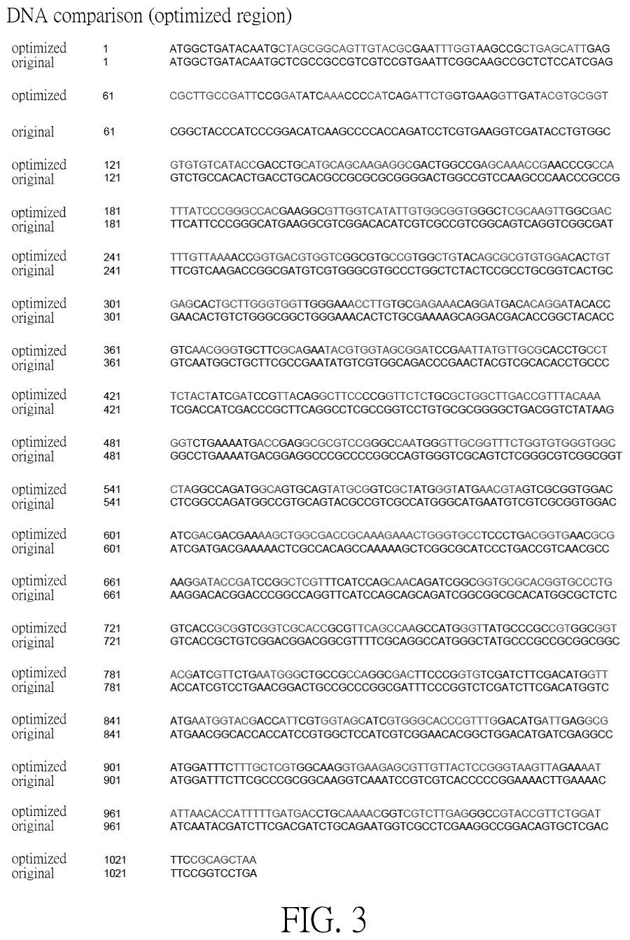

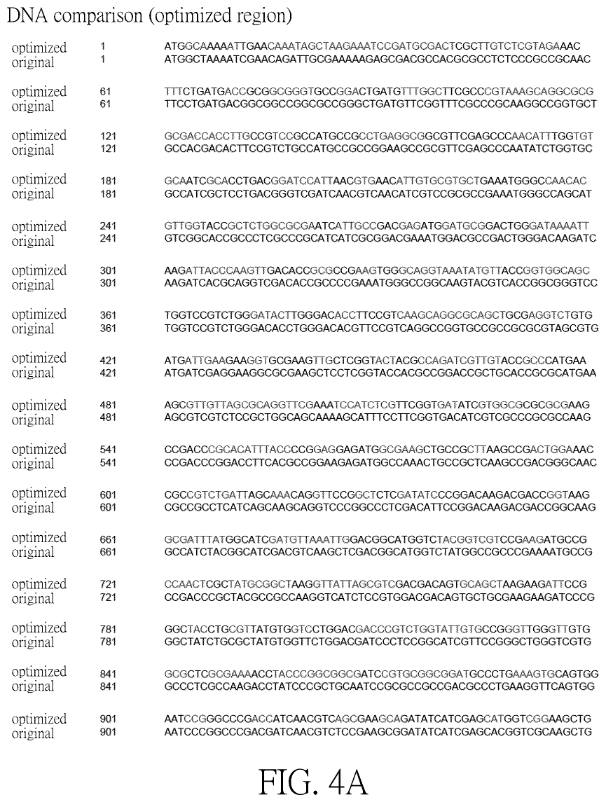

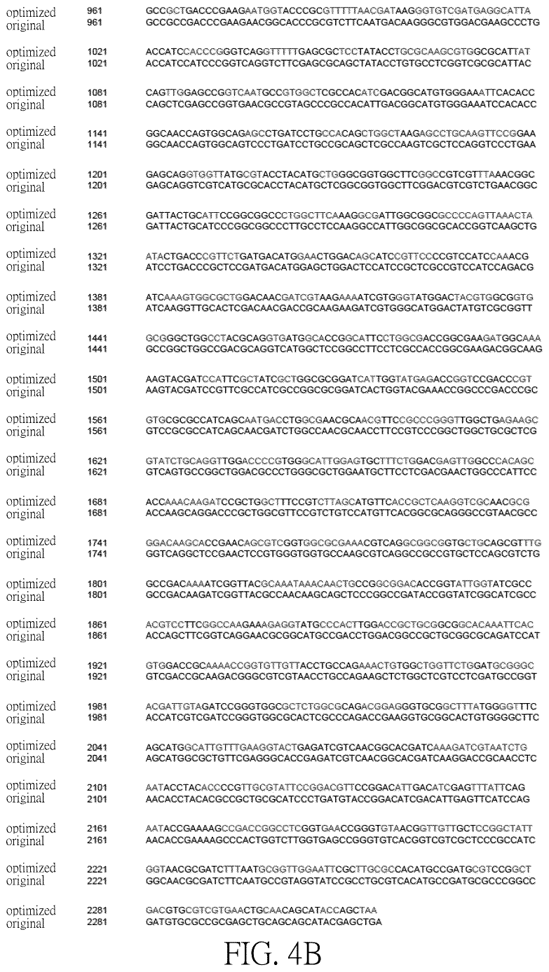

During a gene-to-protein process, each species is affected differently by codons. Although DNA can be transcribed into RNA, a gene cannot be successfully turned into protein without a corresponding tRNA. Hence, by optimizing coding sequences of the alcohol dehydrogenase (EGADH), the aldehyde dehydrogenase (EGAIDH), and the vitreoscilla hemoglobin (VHb) in the present disclosure, the artificially synthesized genetic code identification is suitable for the target strain. In other words, the gene sequences of the present disclosure are already optimized according to the tRNA corresponding to the target strain. Referring to FIG. 3, FIG. 4A, FIG. 4B, and FIG. 5, FIG. 3 is a schematic view showing a gene sequence comparison between the SEQ ID NO: 1 of the present disclosure and the existing alcohol dehydrogenase (EGADH), FIG. 4 (FIG. 4A and FIG. 4B) is a schematic view showing a gene sequence comparison between the SEQ ID NO: 2 of the present disclosure and the existing aldehyde dehydrogenase (EGAIDH), and FIG. 5 is a schematic view showing a gene sequence comparison between the SEQ ID NO: 3 of the present disclosure and the existing vitreoscilla hemoglobin (VHb).

Furthermore, factors that affect codon optimization include differences of ribosome dwell time in the sequences. For optimization of the ribosome dwell time, codons having a low dwell time are sorted. That is, a ribosome will not terminate a codon that is extremely long (which is relevant to a translation speed). Such codon is likely to have a low GC ratio (GC %).

In one embodiment of the present disclosure, a GC % of each of the SEQ ID NO: 1, the SEQ ID NO: 2, and the SEQ ID NO: 3 ranges between 50% and 60%. Specifically, a length of each of an original EGADH sequence and an optimized EGADH sequence is 1,032 bp, a GC % of the original EGADH sequence is approximately 63.57%, and a GC % of the optimized EGADH sequence is approximately 56.2%. A length of each of an original EGAIDH sequence and an optimized EGAIDH sequence is 2,316 bp, a GC % of the original EGAIDH sequence is approximately 64.77%, and a GC % of the optimized EGAIDH sequence is approximately 56.87%. A length of each of an original VHb sequence and an optimized VHb sequence is 441 bp, a GC % of the original VHb sequence is approximately 45.35%, and a GC % of the optimized VHb sequence is approximately 52.83%.

Hydrogen bonds are essential for bonding between adenine (A) and thymine (T) and between guanine (G) and cytosine (C). As such, replication is more stable when bond energy is high, thereby reducing an error rate. In other words, a GC % is relevant to subsequent gene replication and the stability of the gene-to-protein process. Based on the descriptions above, the optimized gene sequence of the present disclosure has a specific GC %, so as to maintain both the ribosome dwell time and the stability of transcription and translation.

In step S20, introduction of the plasmid into the strain can be carried out by a gene gun, electroporation, microinjection, chemical transformation, etc. Preferably, the plasmid is introduced into the strain by the electroporation or the chemical transformation. The electroporation is to apply an electrical current to the strain within an extremely short period of time (from microseconds to milliseconds), so that the strain is subjected to a high voltage and a low capacitance. When a potential difference in a cell membrane is formed, changes occur to the structure of the cell membrane. As a result, the cell membrane is compressed and thinned, thereby generating numerous tiny holes. These tiny holes allow the plasmid to pass through the cell membrane and enter cells of the strain. In one embodiment of the present disclosure, for the electroporation, the strain is preferably processed for 10 msec at a voltage of 0.5 kV, is more preferably processed for 5 msec at a voltage of 1.5 kV, and is most preferably processed for 5 msec at a voltage of 1.0 kV, so as to obtain a large colony count. In another embodiment of the present disclosure, a MicroPulser Electroporator is used for the electroporation, and the electroporation is carried out according to a standard protocol (high efficiency electroporation of E. coli in Section 5) of BIO-RAD.

The chemical transformation is to use a vector for carrying the plasmid and enable an exogenous gene to enter the cell by direct membrane penetration or membrane fusion. For example, the vector can be a liposome for encapsulation of exogenous DNA. After being introduced into the cell, the liposome can merge with the cell membrane, and the exogenous DNA within the liposome can be released into the cell. In the present disclosure, the modified strain is able to at least produce the alcohol dehydrogenase and the aldehyde dehydrogenase, so that the carbon source can be converted into the glycolic acid by one single strain.

In step S30, the carbon source is provided to the modified strain, so that the modified strain converts the carbon source into the glycolic acid. In the present disclosure, the carbon source can be ethylene glycol. Specifically, the ethylene glycol can be ethylene glycol generated from conversion of carbon dioxide by modified cyanobacteria, ethylene glycol generated from polyethylene terephthalate (PET) recycling and degradation (when the degradation is carried out with the ethylene glycol, waste PET can be degraded into dimethyl terephthalate and ethylene glycol), and ethylene glycol generated from any chemical engineering process.

Furthermore, the modified strain of the present disclosure can produce the alcohol dehydrogenase and the aldehyde dehydrogenase. In this way, the ethylene glycol can be converted into glycolaldehyde, and the glycolaldehyde can be further converted into the glycolic acid. The process of converting the ethylene glycol into the glycolic acid by the modified strain of the present disclosure is as shown in Formula 1 below.

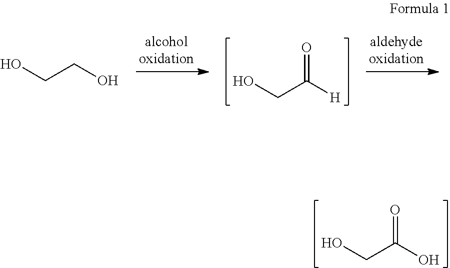

### First Embodiment

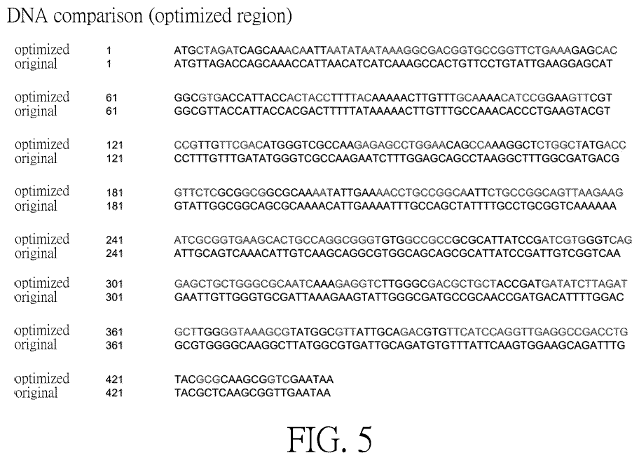

In the first embodiment of the present disclosure, Escherichia coli (E. coli) is used as a strain for modification, and constructed plasmids pAAL-122 and pAALV-122 are introduced into an E. coli host strain DH5a by chemical transformation. The difference between pAAL-122 and pAALV-122 is that pAAL-122 does not include a gene sequence of VHb. Then, strain screening is carried out with antibiotics (lysogeny broth (LB) agar plates containing 30 mg/mL of kanamycin), so as to screen a modified strain that obtains SEQ ID NO: 1, SEQ ID NO: 2, and SEQ ID NO: 3 after success in homologous exchange. One single strain picked from a solid culture medium is placed in 4 mL of a culture medium of LB+kanamycin, and is subjected to shake culture at 170 rpm in a 37° C. incubator. The next day, OD₆₀₀ of an initial strain concentration is adjusted to be approximately 0.05 for expansion culture in 50 mL of a culture medium, and the OD₆₀₀ of the strain concentration is sampled and analyzed relative to time. When the strain is in an initial logarithmic phase of growth (24 h), 50 g/L of ethylene glycol is added for a conversion test. A conversion amount of glycolic acid is analyzed by high-performance liquid chromatography (HPLC), and the results are as shown in FIG. 6.

### Second Embodiment

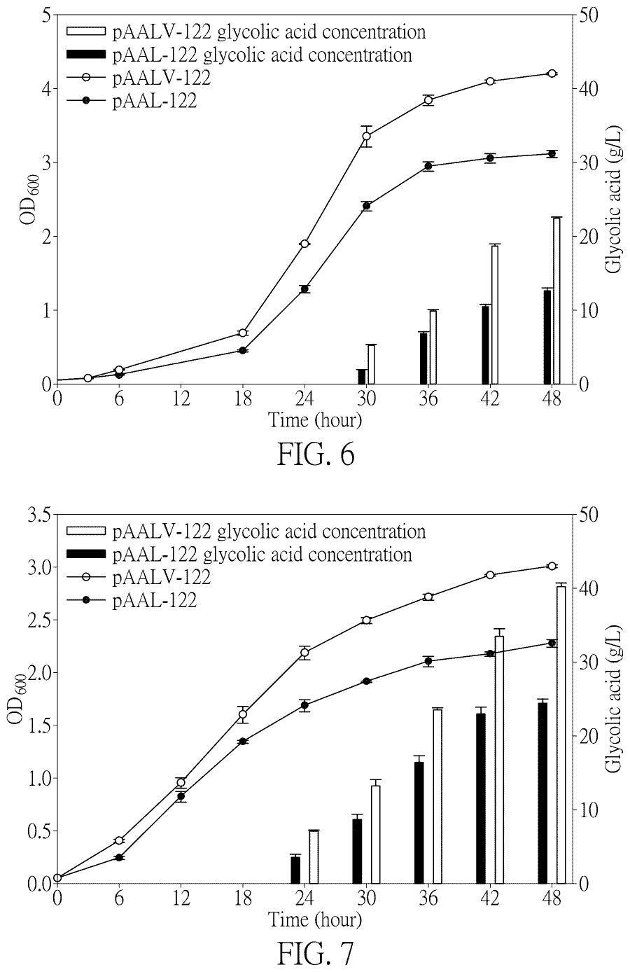

In the second embodiment of the present disclosure, Gluconobacter sp. are used as a strain for modification, and the constructed plasmids pAAL-122 and pAALV-122 are introduced into the Gluconobacter sp. by electroporation. Then, strain screening is carried out with a YPS solid culture medium (5 g/L of a yeast extract, 3 g/L of peptone, 60 g/L of sorbitol, 5 g/L of (NH₄)₂SO₄, and 5 g/L of MgSO₄·7H₂O) containing 5 mg/mL of kanamycin. One single strain picked from a solid culture medium is placed in 4 mL of a culture medium of YPS+kanamycin, and is subjected to shake culture at 170 rpm in a 28° C. incubator. The next day, OD₆₀₀ of an initial strain concentration is adjusted to be approximately 0.05 for expansion culture in 50 mL of a culture medium, and the OD₆₀₀ of the strain concentration is sampled and analyzed relative to time. After twenty-four hours, 50 g/L of ethylene glycol is added for the conversion test. The conversion amount of the glycolic acid is analyzed by high-performance liquid chromatography (HPLC), and the results are as shown in FIG. 7.

It is worth mentioning that the modified strain of the present disclosure has an ability to produce vitreoscilla hemoglobin. As shown in Table 1 below, an expression level of the strain with respect to an EGADH and an EGAIDH can be improved by a transgenic hemoglobin gene (VHb), thereby increasing a yield rate of converting the ethylene glycol into the glycolic acid. In the present disclosure, the yield rate represents an amount of products that can be converted and produced by a culture medium per liter for each time unit, and a conversion rate represents a number of moles for conversion production divided by a number of moles for an initial raw material. Without specific description, the percent sign “%” mentioned in the present disclosure represents “mol %.”

TABLE 1

| | pAAL-122 | pAALV-122 |
|---|---|---|
| E. coli | Yield 12.6 ± 0.3 g/L | Yield 22.46 ± 0.1 g/L |
| | (24 h) | (24 h) |
| | Yield rate 0.525 g/L · h | Yield rate 0.935 g/L · h |
| | Conversion rate 25.2% | Conversion rate 44.9% |
| Gluconobacter | Yield 24.2 ± 0.4 g/L | Yield 39.85 ± 0.35 g/L |
| sp. | (24 h) | (24 h) |
| | Yield rate 1.01 g/L · h | Yield rate 1.66 g/L · h |
| | Conversion rate 48.4% | Conversion rate 79.7% |

The plasmid pAAL-122 contains the EGADH and the EGAIDH, but is without the VHb. The plasmid pAALV-122 contains the EGADH, the EGAIDH, and the VHb.

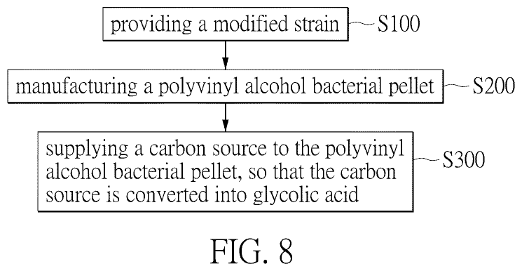

Furthermore, in the present disclosure, an efficiency of forming the glycolic acid by oxidation of the ethylene glycol can be improved by immobilization of the modified strain. As shown in FIG. 8, a method for producing glycolic acid by immobilization includes: providing a modified strain (step S100), in which the modified strain includes gene sequences of SEQ ID NO: 1, SEQ ID NO: 2, and SEQ ID NO: 3; manufacturing a polyvinyl alcohol (PVA) bacterial pellet (step S200), in which the polyvinyl alcohol bacterial pellet contains the modified strain; and supplying a carbon source to the polyvinyl alcohol bacterial pellet, so that the carbon source is converted into the glycolic acid by the modified strain (step S300). The process for manufacturing the polyvinyl alcohol bacterial pellet includes preparing a 1% to 20% polyvinyl alcohol solution. Preferably, a 10% to 15% polyvinyl alcohol solution is prepared.

An operational process for modified strain immobilization is as illustrated in a third embodiment to a sixth embodiment. Strains adopted in the third embodiment to the sixth embodiment are transgenic strains of Gluconobacter sp. pAALV-122.

### Third Embodiment

In the third embodiment of the present disclosure, the operational process for modified strain immobilization is to weigh 2 g of polyvinyl alcohol and 16 g of water for preparation of a 10% PVA solution. In order to ensure dissolution of the PVA, the 10% PVA solution is heated to more than 80° C. for thirty minutes. After the PVA is completely dissolved in the water, the temperature is decreased to 37° C. By adding 2 g of a concentrated bacterial liquid (its cell concentration is a cell dry weight (CDW) of 17.32 g/L) into the PVA solution, a 10% PVA bacterial solution can be obtained. At this time, based on the PVA solution being 100%, a concentration of the added bacterial liquid is 10%. The higher a strain amount is, the better a conversion efficiency is. In one exemplary embodiment, the concentration of the bacterial liquid can also be prepared as 15%. In addition, 120 mL of a 4% saturated boric acid solution and 50 mL of a 0.5 M phosphoric acid solution (Na₂HPO₄ and NaH₂PO₄) are prepared. Then, the 10% PVA bacterial solution is slowly dripped into the 4% saturated boric acid solution by a 10 mL syringe, and undergoes solidification for one hour before a removal and draining process. The 0.5 M phosphoric acid solution is further poured in and let stand for one hour, so as to obtain a 10% PVA bacterial pellet. Said bacterial pellet is directly placed in the phosphoric acid solution at 4° C. for preservation.

### Fourth Embodiment

In the fourth embodiment of the present disclosure, the operational process for modified strain immobilization is to weigh 2 g of the polyvinyl alcohol, 0.2 g of sodium alginate (SA), and 15.8 g of the water for preparation of a 10% PVA-1% SA solution. In order to ensure dissolution of the PVA-SA, the 10% PVA-1% SA solution is heated to more than 80° C. for thirty minutes. After the PVA-SA is completely dissolved in the water, the temperature is decreased to 37° C. By adding 2 g of the concentrated bacterial liquid (its cell concentration is a cell dry weight (CDW) of 17.32 g/L) into the 10% PVA-1% SA solution, a 10% PVA-1% SA bacterial solution can be obtained. In addition, 120 mL of a mixture containing 4% saturated boric acid and 1% calcium chloride, and 50 mL of the 0.5 M phosphoric acid solution (Na₂HPO₄ and NaH₂PO₄) are prepared. Then, the 10% PVA-1% SA bacterial solution is slowly dripped into the mixture containing 4% saturated boric acid and 1% calcium chloride by the 10 mL syringe, and undergoes solidification for one hour before the removal and draining process. The 0.5 M phosphoric acid solution is further poured in and let stand for one hour, so as to obtain a 10% PVA-1% SA bacterial pellet. Said bacterial pellet is directly placed in the phosphoric acid solution at 4° C. for preservation.

### Fifth Embodiment

In the fifth embodiment of the present disclosure, the operational process for modified strain immobilization is to weigh 0.3 g of the sodium alginate and 17.7 g of the water for preparation of a 1.5% SA solution. In order to ensure dissolution of the SA, the 1.5% SA solution is heated to more than 80° C. for thirty minutes. After the SA is completely dissolved in the water, the temperature is decreased to 37° C. By adding 2 g of the concentrated bacterial liquid (its cell concentration is a cell dry weight (CDW) of 17.32 g/L) into the 1.5% SA solution, a 1.5% SA bacterial solution can be obtained. In addition, 120 mL of 2% calcium chloride is prepared. Then, the 1.5% SA bacterial solution is slowly dripped into a solution of the 2% calcium chloride by the 10 mL syringe, and undergoes solidification for one hour. Afterwards, a bacterial pellet is washed and placed in an aqueous solution at 4° C. for preservation.

### Sixth Embodiment

In the sixth embodiment of the present disclosure, the operational process for modified strain immobilization is to weigh 2 g of the polyvinyl alcohol and 16 g of the water for preparation of the 10% PVA solution. In order to ensure dissolution of the PVA, the 10% PVA solution is heated to more than 80° C. for thirty minutes. After the PVA is completely dissolved in the water, the temperature is decreased to 37° C. By adding 2 g of the concentrated bacterial liquid (its cell concentration is a cell dry weight (CDW) of 17.32 g/L) into the PVA solution, the 10% PVA bacterial solution can be obtained. The 10% PVA bacterial solution is poured into a mold, is frozen at −20° C. for two hours to twelve hours according to the size of the mold, and is taken out and thawed in a water bath at a room temperature for thirty minutes to sixty minutes. This process is repeated five times for formation of a bacterial mass. Finally, the bacterial mass is thawed, sliced, and preserved at 4° C.

Immobilized particles of the modified strains from the third embodiment to the sixth embodiment are obtained based on the same wet weight, and are tested according to different hydraulic retention time (HRT). A feeding material is 50 g/L of an ethylene glycol solution+5% YPS (5 g/L of a yeast extract, 3 g/L of peptone, 60 g/L of sorbitol, 5 g/L of (NH₄)₂SO₄, and 5 g/L of MgSO₄·7H₂O), a total reaction volume is 400 ml, and air is introduced at 0.5 vvm. The unit “vvm” represents a ratio of a volume of air to a volume of medium per minute. The hydraulic retention time represents an average amount of time during which a liquid to be reacted remains in a reactor, and involves dividing a volume by an influent flow rate. In the present embodiment, the liquid to be reacted refers to the ethylene glycol solution, and the air refers to an oxygen-containing gas.

TABLE 2

| | Yield rate (g/L · h) | | |
|---|---|---|---|
| Embedding manner | Batch | HRT 16.7 h | HRT 10 h |
| Third embodiment | 1.02 ± 0.22 | 0.89 ± 0.11 | 0.71 ± 0.23 |
| (10% PVA) | | | |
| Fourth embodiment | 1.34 ± 0.13 | 1.21 ± 0.23 | 1.03 ± 0.12 |
| (10% PVA-1% SA) | | | |
| Fifth embodiment | 0.89 ± 0.09 | 0.65 ± 0.15 | 0.51 ± 0.02 |
| (1.5% SA) | | | |
| Sixth embodiment | 1.61 ± 0.34 | 1.57 ± 0.23 | 1.24 ± 0.11 |
| (frozen 10% PVA) | | | |

Referring to Table 2 above, the third embodiment to the fifth embodiment are compared. Compared with a single use of the PVA or the SA in the third embodiment or the fifth embodiment, a mixed use of the 10% PVA-1% SA in the fourth embodiment has a greater yield rate. Here, the yield rate represents an amount of the glycolic acid that can be produced per unit time and unit volume. That is to say, an efficiency of converting the ethylene glycol into the glycolic acid can be increased when the PVA and the SA are used in a mixed manner. It is worth mentioning that, under different process operations, the efficiency of converting the ethylene glycol into the glycolic acid may also be affected. A comparison can be made between the third embodiment and the sixth embodiment. Since the bacterial mass manufactured from a freeze-thaw cycle does not need to be immersed in a solution of boric acid and phosphoric acid, its cells are less harmed. As a result, when the 10% PVA is similarly used, the efficiency of converting the ethylene glycol into the glycolic acid by the bacterial mass manufactured from the freeze-thaw cycle is the highest.

### Beneficial Effects of the Embodiments

In conclusion, in the method for producing the glycolic acid by immobilization provided by the present disclosure, by virtue of “providing a modified strain, in which the modified strain includes gene sequences of SEQ ID NO: 1, SEQ ID NO: 2, and SEQ ID NO: 3” and “manufacturing a polyvinyl alcohol bacterial pellet, in which the polyvinyl alcohol bacterial pellet contains the modified strain,” the technology of strain immobilization can be used to improve the efficiency of forming the glycolic acid by oxidation of the ethylene glycol.

For the modified strain of the present disclosure, the expression level of the modified strain with respect to the EGADH and the EGAIDH can be improved by the transgenic hemoglobin gene (VHb), thereby increasing the yield rate of converting the ethylene glycol into the glycolic acid. Since the modified strain of the present disclosure can produce hemoglobin, the yield rate of converting the ethylene glycol into the glycolic acid can be further increased by introducing the air at a range of between 0.3 vvm and 1 vvm during a reaction process.

Furthermore, in biological modification of the present disclosure, the ethylene glycol manufactured from conversion of the carbon dioxide by the cyanobacteria, from the PET recycling and degradation, and from chemical plants is used as a raw material. Based on biotransformation, production and purification processes of the glycolic acid are established. In this way, a glycolic acid raw material having a high purity can be obtained, and the technology of strain immobilization can be used to further improve the efficiency of forming the glycolic acid by oxidation of the ethylene glycol.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for producing glycolic acid by immobilization, comprising:
providing a modified strain, wherein the modified strain includes gene sequences of SEQ ID NO: 1, SEQ ID NO: 2, and SEQ ID NO: 3;
manufacturing a polyvinyl alcohol bacterial pellet, wherein the polyvinyl alcohol bacterial pellet contains the modified strain; and
supplying a carbon source to the polyvinyl alcohol bacterial pellet, so that the carbon source is converted into the glycolic acid by the modified strain; wherein the polyvinyl alcohol bacterial pellet is mixed with an auxiliary solution;
wherein air is introduced when the carbon source is supplied to the polyvinyl alcohol bacterial pellet.

2. The method according to claim 1, wherein manufacturing the polyvinyl alcohol bacterial pellet includes:
preparing a 1% to 20% polyvinyl alcohol solution, and adding the modified strain into the polyvinyl alcohol solution for uniform mixing, so as to form a polyvinyl alcohol bacterial solution;
preparing a saturated boric acid solution and a phosphoric acid solution;
dripping the polyvinyl alcohol bacterial solution into the saturated boric acid solution, so as to obtain a semi-finished product of the polyvinyl alcohol bacterial pellet; and
taking out the semi-finished product of the polyvinyl alcohol bacterial pellet for draining, and pouring in the phosphoric acid solution and letting stand for a predetermined time, so as to obtain the polyvinyl alcohol bacterial pellet.

3. The method according to claim 2, wherein manufacturing the polyvinyl alcohol bacterial pellet further includes preparing 0.5% to 1% sodium alginate.

4. The method according to claim 2, wherein the predetermined time ranges between thirty minutes and two hours.

5. The method according to claim 2, wherein a concentration of the saturated boric acid solution ranges between 1% and 5%, and a concentration of the phosphoric acid solution ranges between 0.1 M and 1 M.

6. The method according to claim 1, wherein the carbon source is ethylene glycol, and an aeration volume of the air ranges between 0.3 vvm and 1 vvm.

7. The method according to claim 1, wherein the modified strain is modified Escherichia coli, modified Gluconobacter sp., modified Rhodococcus, or modified oxidative bacilli.

8. The method according to claim 1, wherein the auxiliary solution is formed by a yeast extract, peptone, sorbitol, (NH₄)₂SO₄, and MgSO₄·7H₂O.

9. The method according to claim 1, wherein a GC % of each of the SEQ ID NO: 1, the SEQ ID NO: 2, and the SEQ ID NO: 3 ranges between 50% and 60%.

10. The method according to claim 1, wherein the modified strain has an ability to produce an alcohol dehydrogenase, an aldehyde dehydrogenase, and vitreoscilla hemoglobin.
